(19) United States
(12) Patent Application Publication
(10) Pub. No.: US 2016/0324720 A1
(43) Pub. Date: Nov. 10, 2016

(54) HEATED AND VIBRATING PILLOW

(71) Applicant: Karen Lear, Burbank, CA (US)

(72) Inventor: Karen Lear, Burbank, CA (US)

(21) Appl. No.: 14/703,758

(22) Filed: May 4, 2015

(51) Int. Cl. A61H 23/02; A47G 9/10; A61F 7/00

(52) U.S. Cl. CPC A61H 23/02; A61F 7/007; A47G 9/1036

(57) ABSTRACT

A heating and vibrating pillow for providing therapeutic heating and massaging functions through a conventionally sized and shaped bed pillow. The heating and vibrating pillow comprises a pillow housing filled with filling material such that a vibrating encasement is suspended therein. The surface of one side of the pillow housing includes an electrical switch operated flexible heating mesh configured to generate a heated area on the surface of the pillow housing. The vibrating encasement includes an electrical switch operated vibrator that is configured to generate vibrations from the center of the pillow. The heating and vibrating pillow is wired to receive electricity from either a electrical plug an internal battery.

### BACKGROUND OF THE INVENTION

1. Field of the Invention

This invention relates generally to cushioned support structures and, more particularly, to a pillow having selectively operative heating and vibrating elements.

2. Description of the Prior Art

The use and design of pillows as cushioned support structures for the head (or other body parts) is well known. In some circumstances, pillows and other cushions have been adapted with heating and/or vibrating components in effort to promote relaxation, provide added comfort, or otherwise configure the pillow to be more suitable for a targeted purpose. A problem which still exists, however, is that existing pillows with a vibrating and/or heating capacity lose their pliability and are thus provide less comfort that conventional pillows. Thus, there remains a need for a heating and vibrating pillow that retains its pliability while offering heating and vibrating functions. It would be helpful if such a heating and vibrating pillow defined an enclosed pillow having surface interface control for its internal electrical components. It would be additionally desirable for such a heating and vibrating pillow to include an electrical power adapter that enabled operation from wall electrical jacks or internal batteries.

The Applicant's invention described herein provides for a heating and vibrating pillow adapted to allow a user to apply heat and/or vibration therapy to their head in effort to promote relaxation or relieve pain. The primary components in Applicant's heating and vibrating pillow are a pillow housing, a vibrating encasement, and filling material. When in operation, the heating and vibrating pillow enables the selectable application of heat and/or vibration through a conventionally sized and shaped pillow. As a result, many of the limitations imposed by prior art structures are removed.

### SUMMARY OF THE INVENTION

A heating and vibrating pillow for providing therapeutic heating and massaging functions through a conventionally sized and shaped bed pillow. The heating and vibrating pillow comprises a pillow housing filled with filling material such that a vibrating encasement is suspended therein. The surface of one side of the pillow housing includes an electrical switch operated flexible heating mesh configured to generate a heated area on the surface of the pillow housing. The vibrating encasement includes an electrical switch operated vibrator that is configured to generate vibrations from the center of the pillow. The heating and vibrating pillow is wired to receive electricity from either a electrical plug an internal battery.

It is an object of this invention to provide an extension bar for a heating and vibrating pillow that retains its pliability while offering heating and vibrating functions.

It is another object of this invention to provide a heating and vibrating pillow that defined an enclosed pillow having surface interface control for its internal electrical components.

It is yet another object of this invention to provide a heating and vibrating pillow that includes an electrical power adapter that enabled operation from wall electrical jacks or internal batteries.

These and other objects will be apparent to one of skill in the art.

### BRIEF DESCRIPTION OF THE DRAWINGS

FIG. 1 is a top plan view of a heating and vibrating pillow built in accordance with the present invention.

FIG. 2 is a bottom plan view of a heating and vibrating pillow built in accordance with the present invention with the vibrating encasement and battery housing in shadow.

FIG. 3 is a top plan view of a vibrating encasement of a heating and vibrating pillow built in accordance with the present invention.

### DETAILED DESCRIPTION OF THE INVENTION

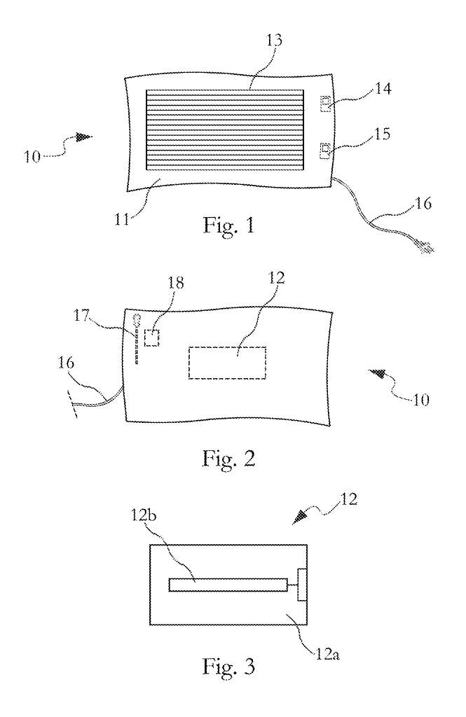

Referring now to the drawings and in particular FIGS. 1 and 2, a heating and vibrating pillow 10 is shown having a pillow housing 11 and a vibrating encasement 12. The pillow housing 11 defines an enclosure sized and shaped as conventional pillows for use on a bed. The surface of the pillow housing 11 includes a flexible heating mesh 13 operatively coupled with a heating switch 14 such that the heating switch 14 enables electricity to be selectively provided thereto. In the preferred embodiment, the heating mesh 13 defines a mesh of Polyester filament and conductive fiber.

In addition to the heating mesh 13 and heating switch 14, the pillow housing 11 additionally includes a vibrating switch 15, an electrical cord 16, and a battery compartment 17. The vibrating switch 15 operatively coupled with the vibrating encasement 12 such that the vibrating switch 15 enables electricity to be selectively provided to the vibrating encasement 12. The electrical cord 16 defines a conventional electrical cord for connecting to an electrical wall outlet so as to avail electricity from the wall outlet to the heating and vibrating pillow 10. The battery compartment 17 defines a closeable section for receiving a conventional battery 18 and availing electricity therefrom to the heating and vibrating pillow 10. It is understood that in the preferred embodiment, the heating and vibrating pillow 10 includes electrical wiring that causes it to operate of electricity from a wall outlet when available and operate from electricity from a battery 18 when electricity from a wall outlet is not available. Such electrical wiring additionally connects to the heating mesh 13 and the vibrating encasement 12, thereby facilitating the selective provision of electricity to these components from the electrical cord 16 or battery 18.

In the preferred embodiment, the internal vibrating encasement 12 defines a rubber housing 12a having a vibrator 12b encased therein. The vibrating encasement 12 includes an electrical input 12c for connecting to the electrical wiring in the heating and vibrating pillow 10 to allow the vibrating encasement 12 to receive electricity from the electrical components of the heating and vibrating pillow 10 (selectively provided by operation of the vibrating switch 15). It is contemplated that the vibrator 12b is operative to vibrate a several distinct speeds and the vibrations generated by the vibrator 12b cause the entire vibrating encasement 12 to shake.

In the preferred embodiment, conventional pillow filling material (not shown) is dispersed throughout the inside of the pillow housing 11. It is contemplated that the vibrating encasement 12 is positioned in the pillow housing 11 such that it is surrounded by filling material, essentially suspended in a central location in the pillow housing 11 by the filling material. Accordingly, it is appreciated that the pillow housing 11 remains substantially pliable such that it can be depressed or bent as conventional pillows except in the central area corresponding to where the vibrating encasement 12 is suspended. It is contemplated that most conventional filling materials, including polyester, foam, feather, wool, hair or rubber may be employed in accordance with the present invention.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A heating and vibrating pillow, comprising:
a pillow housing defining an enclosure having a top side and a bottom side, wherein said pillow housing includes a power source defining at least one of an electrical cord operative to receive electricity from a conventional wall electrical outlet and a battery compartment operative to receive electricity from an integrated battery;
said top side having a flexible heating mesh integral therewith, wherein the flexible heating mesh is configured to selectively receive electricity from said power source; and
a vibrating element disposed inside said pillow housing, wherein the vibrating element is configured to selectively receive electricity from said power source.

2. The heating and vibrating pillow of claim 1, wherein said flexible heating mesh is configured to selectively receive electricity from said power source through a manual heating switch accessible from the top side of said pillow housing.

3. The heating and vibrating pillow of claim 2, wherein said vibrating element is configured to selectively receive electricity from said power source through a manual vibrating switch accessible from the top side of said pillow housing.

4. The heating and vibrating pillow of claim 3, wherein said vibrating element is encased in a vibrator housing suitable to disperse the movement of the vibrating element over a larger area.

5. The heating and vibrating pillow of claim 4, wherein said pillow housing includes filling material disposed therein and configured such that the vibrating element and vibrator housing are suspended in the pillow housing by the filling material.

6. The heating and vibrating pillow of claim 1, wherein the power source includes power source the electrical cord and battery compartment and is configured to selectively provide electricity from either the electrical cord or battery compartment at any given moment.

7. The heating and vibrating pillow of claim 1, wherein said vibrating element is configured to selectively receive electricity from said power source through a manual vibrating switch accessible from the top side of said pillow housing.

8. The heating and vibrating pillow of claim 1, wherein said vibrating element is encased in a vibrator housing suitable to disperse the movement of the vibrating element over a larger area.

9. The heating and vibrating pillow of claim 8, wherein said pillow housing includes filling material disposed therein and configured such that the vibrating element and vibrator housing are suspended in the pillow housing by the filling material.
